(19) United States
(12) Patent Application Publication
(10) Pub. No.: US 2014/0076317 A1
(43) Pub. Date: Mar. 20, 2014

(54) PULSE OXIMETRY IN RESPIRATORY THERAPY PATIENT INTERFACE

(71) Applicants: Gary William Lotz, Mars, PA (US); Elias George Diacopoulos, Export, PA (US); Douglas Mechlenburg, Murrysville, PA (US); Winslow Kevin Duff, Export, PA (US)

(72) Inventors: Gary William Lotz, Mars, PA (US); Elias George Diacopoulos, Export, PA (US); Douglas Mechlenburg, Murrysville, PA (US); Winslow Kevin Duff, Export, PA (US)

(73) Assignee: KONINKLIJKE PHILIPS N.V., EINDHOVEN (NL)

(21) Appl. No.: 14/006,469

(22) PCT Filed: Mar. 15, 2012
(86) PCT No.: PCT/IB2012/051221

§ 371 Date: Nov. 8, 2013

Related U.S. Application Data
(60) Provisional application No. 61/466,488, filed on Mar. 23, 2011.

(51) Int. Cl. A61B 5/00; A61M 16/06; A61M 16/08; A61M 16/00; A61B 5/1455

(52) U.S. Cl. CPC A61B 5/4836; A61M 16/0051; A61M 16/0057; A61B 5/14551; A61M 16/0875; A61M 16/06
USPC 128/204.23

(57) ABSTRACT

Provided are systems and methods for utilizing blood oxygenation information with respiratory therapy. These systems and methods may provide respiratory therapy to a patient (119), which may include providing a flow of gas to a patient via a patient interface. Blood oxygenation information for the patient is then obtained. The blood oxygenation information is then used to adjust the respiratory therapy, advance a diagnosis for the patient, or for other purposes.

The present disclosure pertains to systems and methods for using blood oxygenation information with respiratory support, and, in particular, the integration of a blood oxygenation sensor into a patient interface of a respiratory support system.

Delivery of respiratory therapy is often customized for patients based on one or more factors. These factors may include the patient's initial diagnosis, age, weight, physical condition, and/or other factors. However, often times, physicians and other caregivers must utilize their experience and/or trial and error to properly customize respiratory therapy. As such, the use of measurable indicators to guide customization of respiratory therapy may be advantageous. As pulse oximetry data relates to oxygenation of a patient's blood, this data would be useful in tailoring respiratory therapy for a patient, providing diagnostic data in concert with respiratory therapy, and for other uses. Systems with pulse oximetry elements built in would provide additional clinical and logistical advantages. As such, integration of blood oxygenation data into respiratory therapy systems and methods via a sensor integrated into a mask or other patient interface would be advantageous.

Accordingly, it is an object of one or more embodiments of the present invention to provide a respiratory therapy device comprising: a pressure generator that generates a flow of gas; a delivery conduit connected to the pressure generator and having a patient interface, wherein the delivery conduit delivers the flow of gas to a patient connected to the patient interface; a sensor integrated into the patient interface that measures blood oxygenation information for the patient; and a controller that controls the pressure generator, receives the blood oxygenation information for the patient, and initiates an action relating to the patient based on the received blood oxygenation information.

It is yet another aspect of one or more embodiments of the present invention to provide a method for providing respiratory therapy comprising: providing respiratory therapy to a patient using a patient interface of a respiratory therapy system; measuring blood oxygenation information for the patient using a sensor integrated into the patient interface; and initiating an action relating to the patient based on the received blood oxygenation information.

It is yet another aspect of one or more embodiments to provide a respiratory therapy device comprising: a means for generating a flow of gas; a means for delivering the flow of gas to a patient via a patient interface; a means for measuring blood oxygenation information for the patient that is integrated into the patient interface; and a controller means for controlling the means for generating a flow of gas, receiving the blood oxygenation information for the patient, and initiating an action relating to the patient based on the received blood oxygenation information.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

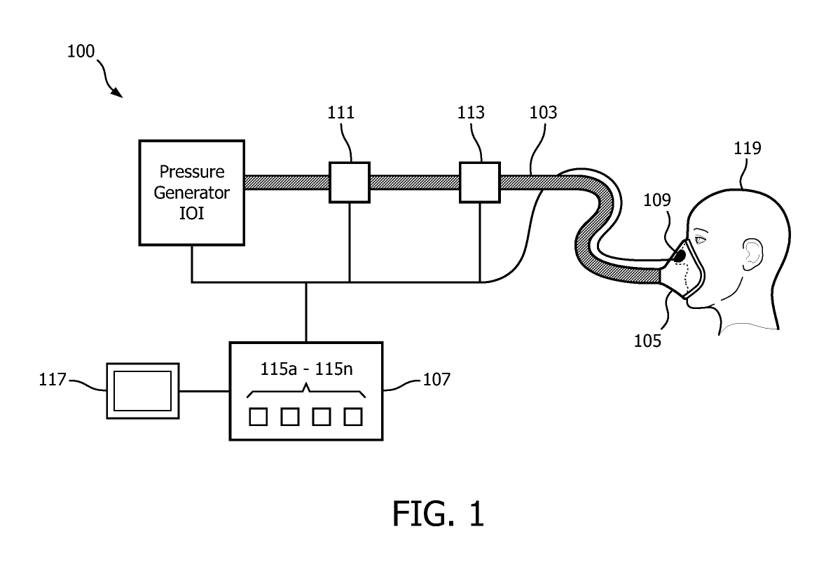

FIG. 1 is an example of a system for using blood oxygenation information with respiratory therapy, according to various embodiments of the invention;

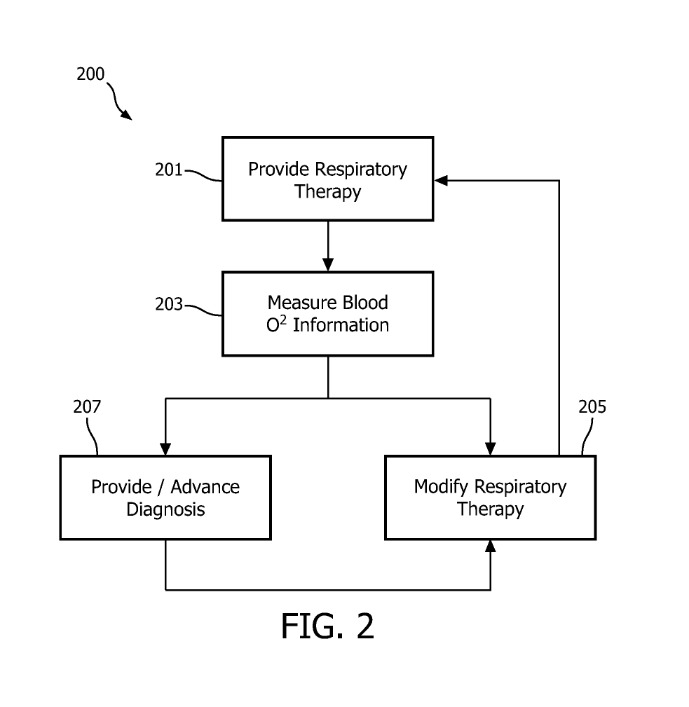

FIG. 2 is an example of a process for using blood oxygenation information with respiratory therapy, according to various embodiments of the invention.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

In some embodiments, systems and methods for utilizing blood oxygenation information with respiratory therapy are provided. FIG. 1 illustrates a system 100, which is an example of a system for using blood oxygenation information with respiratory therapy. System 100 may include a pressure (or flow) generator 101, a delivery conduit 103, a patient interface 105, a controller 107, a blood oxygenation sensor 109, and/or other elements.

Pressure generator 101 may be or include, for example, a blower such as used in a conventional positive airway pressure (PAP) therapy, continuous positive airway pressure (CPAP) therapy, ventilation, or other respiratory therapy device. Pressure generator 101 may receive breathing gas from any suitable source such as, for example, a pressurized tank of oxygen or air, the ambient atmosphere, or a combination thereof. Pressure generator 101, may generate a flow of breathing gas such as, for example, air, oxygen, or a mixture thereof, for delivery to an airway of a patient at a desired pressure. Pressure generator 101 may be used to control the flow and/or pressure of breathing gas within the patient circuit through, for example, variations of the speed of its associated fan and/or via other methods.

Delivery conduit 103 is connected to pressure generator 101 so as to deliver breathing gas to a patient via patient interface 105. Patient interface 105 may include a mask, a nasal cannula, an invasive conduit (inserted via intubation or trans-tracheally) or other patient interface. Delivery conduit 103 and patient interface 105 may be referred to as a “patient circuit.”

Controller 107 may be or include one or more processing devices (e.g., a microprocessor or microcontroller), associated memory, input/output ports, and/or other features enabling receipt and processing of data, performance of calculations, and formulation and issuance of commands for controlling pressure generator 101 and/or other components of system 100.

Blood oxygenation sensor 109 may be or include any sensor that measures blood oxygenation information for a patient. In some implementations, blood oxygenation information may be or include pulse oximetry information, which is a non-invasive method of monitoring the oxygenation of a patient's hemoglobin (i.e., SpO₂). Pulse oximetry is a surrogate measure of more invasive procedures to obtain blood gas measurements such as, for example, arterial, venous, or capillary blood draws which are less desirable for many reasons. In some embodiments, blood oxygenation sensor 109 may comprise a non-invasive sensor that includes a light emitter portion that emits light (e.g., red and infra-red light) onto a skin surface of a patient and a light detector portion positioned so as to detect the emitted light that is reflected from the skin of the patient (i.e., a reflectance-based sensor). In some embodiments, the light detector portion may be positioned so as to detect the emitted light that passes through the skin of a patient (i.e., a transmittance-based sensor). Blood oxygenation sensor 109 then determines the blood oxygenation information based on the light absorbed by the skin of the patient. An example of a blood oxygenation sensor that may be used includes those made by Nonin Medical, Inc. Other blood oxygenation sensors may also be used.

In some embodiments, blood oxygenation sensor 109 may be integrated into patient interface 105. For example, reflectance based blood oxygenation sensors may be placed on a mask interface where full skin contact can be made and maintained consistently such as, on the surface of the nose, the forehead, or on a strap (for example, an earlobe patch or clip may be used when a strap of the patient interface travels over, on, or close to the patient's ear). In some embodiments, transmittance-based blood oxygen sensors may be placed so that light can be emitted onto the skin of a patient 119 and a sensor is positioned so as to sense the light passing therethrough such as, for example, on a nose of the patient (e.g., in a nasal pillow mask) or on an earlobe clip (e.g., on a strap of a patient interface that travels over, on, or near the patient's ear). Other placements of blood oxygenation sensor 109 may be used.

System 100 may also include one or more valves (e.g., valve 111) disposed on the patient circuit (e.g., on delivery conduit 103) that enable control of the flow and/or pressure of gas within the patient circuit (control of pressure generator 101 may also be used to control the flow and/or pressure of gas within the patient circuit). System 100 may also include one or more flow and/or pressure sensors (e.g., sensor 113) disposed on the patient circuit (e.g., on delivery conduit 103) that enable determination of the flow and/or pressure of gas within the patient circuit.

In some embodiments, system 100 may include a display device 117, which may be or include an electronic computer-implemented display such as, for example, a cathode-ray device, a liquid crystal display device, light emitting diode (LED) display device, or other display device. Display device 117 may enable a graphical user interface (GUI) that may be used to present information to a clinician or other use such as, information relating to respiratory therapy, information regarding blood oxygenation levels, information regarding suggested courses of action, information regarding diagnosis of a patient, alerts, warnings, or other information. Display device 117 may also provide an input device (e.g., a touch screen) for clinicians or other users. Other input devices (e.g., a keypad, keyboard, mouse, etc.) may also be used. In some embodiments, information from system 100 may also or alternatively be output to one or more separate and/or remote computing devices or computer applications. For example, in some instances information from system 100 may be sent across a network (which may be or include the Internet) to one or more destinations (e.g., EncoreAnywhere™) and/or may be accessible by clinicians or other users over the internet from one or more websites or other interfaces.

In some embodiments, pressure generator 101, blood oxygenation sensor 105, valve 111, sensor 113, and/or other elements of system 100 are operatively connected to controller 107 such that controller receives information from and issues commands to one or more of these elements. In some implementations, controller 107 may include one or more modules 115a-115n. Modules 115a-115n may include or comprise one or more software programs, computer-executable instructions, and/or data causing the one or more processing devices of controller 107 to perform one or more of the features or functions described herein. For example, modules 115a-115n may include one or more modules that: receive blood oxygenation information from blood oxygenation sensor 109; receive gas flow and/or pressure data for delivery conduit 103 from sensor 113; perform one or more calculations, comparisons, and/or determinations; control pressure generator 101, valve 111, and/or other system components based (at least in part) on the received data (and/or other data); output any received data and/or the results of any calculations, comparisons, or determinations (e.g., via a GUI on display device 117); and/or enable other features or functions. The instructions of modules 115a-115n may be stored on a hard disc, EPROM, EEPROM, other non-volatile memory, or other memory/storage device that is operatively connected to or in communication with the one or more processors of controller 107.

Methods for utilizing blood oxygenation information in relation to pressure support may also be provided. FIG. 2 illustrates a process 200, which is an example of a process for utilizing blood oxygenation information with respiratory therapy. Process 200 includes an operation 201, wherein a patient is provided with respiratory therapy. Respiratory therapy may include positive airway pressure (PAP) therapy, continuous, bi-level, or variable positive airway pressure therapy (CPAP, BiPAP, or servoventilation, respectively), ventilation, or other respiratory therapy. In some embodiments, respiratory therapy may be provided to the patient using the systems described herein such as, for example, system 100. For example, controller 107 may, according to a set of instructions (e.g., one of modules 115a-115n), issue commands to pressure generation 101, valve 111 and/or other components so as to provide the respiratory therapy to a patient via the patient circuit.

Process 200 may include an operation 203, wherein blood oxygenation information for the patient is received. For example, an SpO₂ measurement (i.e., the saturation of oxygen in a patient's hemoglobin) may be obtained using a blood oxygenation sensor (e.g., 109). This blood oxygenation information may then be sent to/received by a controller (e.g., controller 107). In one example, a module from modules 115a-115n may enable receipt of the SpO₂ measurement.

In some embodiments, process 200 may proceed from operation 203 to an operation 205, wherein the respiratory therapy is modified according to the received blood oxygenation information. Lower than expected SpO₂ levels may be an indication that a given therapy level is not adequate and that additional pressure is needed to maintain an open airway. For example, SpO₂ levels lower than a given threshold may trigger a modification in a pressure component of respiratory therapy (e.g., an increase in the pressure, in an attempt to maintain open airways and therefore raise SpO₂ levels in the patient). In another example, lower than expected SpO₂ levels may trigger an increase in the breathing frequency for a patient so as to raise SpO₂ levels. Conversely, SpO₂ levels higher than a threshold level, may also trigger a modification in the breathing frequency of the pressure therapy (e.g., a decrease in the frequency) that results in a consistent SpO₂ level and stabilization of the patient's breathing . Other modifications may be made based on received SpO₂ levels.

In another example, lower than expected SpO₂ levels may be an indication that a current mode of therapy (e.g., CPAP) is not adequately supporting the patient's respiratory needs, for example, that the patient may require a more sophisticated therapy (e.g., auto servo ventilation).

SpO₂ levels may also supply information related to Heart Rate Variability (HRV), or other cardiac measurements, which may be an indicator of patient/device synchrony, and/or patient tolerance, to a particular therapy or mode of therapy. With this information, alternative solutions may be assessed and/or proposed by the device (e.g., via logic provided by controller 107 or one of modules 115a-115n) or following clinician evaluation.

In another example, if a patient's SpO₂ levels were to drop below a certain threshold for a given period of time, (acutely or cumulatively), despite receiving adequate therapy to treat the a disorder (e.g., pressure for a sleep disorder), then a message can be generated for display to a clinician (or other user) to add supplemental oxygen to support the patient. If there is currently supplemental oxygen added to the circuit, but the amount is not providing adequate oxygenation, an increase may be suggested.

In some implementations, the modification of operation 203 may be enabled by one or more modules 115a-115n of controller 107. For example, one or more of the modules may store one or more threshold levels, look up tables, logic, and/or other instructions for modifications based, at least in part, on SpO₂ measurements and/or other input.

In some embodiments, after modification of the respiratory therapy in operation 205, process 200 may return to operation 201 wherein the modified respiratory therapy is provided to the patient. Further blood oxygenation measurements and subsequent modifications may then be made. Accordingly, as provision of therapy and blood oxygenation monitoring is continued after modification, blood oxygenation information may be used to provide a feedback loop for control of respiratory therapy.

In some embodiments, process 200 may proceed from operation 203 to an operation 207, wherein the received blood oxygenation levels are used, alone or in concert with other data, to provide or advance a diagnosis of the patient. For example, the patient may have had a baseline SpO₂ level recorded prior to beginning a certain type of respiratory therapy. If, upon provision of this specific type of therapy, SpO₂ levels do not change in an expected manner, a diagnosis of a certain pathology may be ruled out. Conversely, if, upon provision of this specific type of therapy, SpO₂ levels change in an expected/favorable manner, then a diagnosis of a certain pathology may be advanced or confirmed. As mentioned above, SpO₂ levels could indicate whether a given therapy is appropriate. Intermittent hypoxia may be a cause of long-term co-morbidities (e.g., stroke, heart failure, gastroesophageal reflux disease [GERD], diabetes, etc.). As reimbursement policies may state that certain reduced SpO₂ levels (e.g., <88%) for more than 10 minutes qualifies the patient for supplemental oxygen, feedback from a blood oxygen sensor may be valuable. In this manner, the use of SpO₂ data in concert with therapy, and the use of apparatuses for this purpose, can be beneficial.

In some embodiments, these diagnostic features may be enabled by one or more modules 115a-115n of controller 107. For example, may include one or more threshold levels, look up tables, algorithms, and/or other instructions for outputting diagnostically relevant information based, at least in part, on SpO₂ measurements and/or other input. In some implementations, one or more modules 115a-115n may enable output (e.g., display on a screen/GUI of display device 117, output to a separate device or application as discussed herein, or otherwise output) of SpO₂ measurements enabling other devices and/or clinicians to draw diagnostic conclusions.

In some embodiments, after a diagnosis is provided or advanced in operation 207, process 200 may proceed to operation 205, wherein respiratory therapy is modified in light of the diagnosis. Process 200 may then return to operation 201 wherein the modified respiratory therapy is provided to the patient. Further blood oxygenation measurements and subsequent modifications may then be made. Again, this continued provision of therapy and blood oxygenation monitoring after diagnosis and modification provides for a feedback loop for operation of respiratory therapy.

In some embodiments, tangible computer-readable media comprising computer-executable instructions for causing one or more computer processors to perform one or more of the features and functions set forth herein, including the operations of the methods described herein, may be provided.

The systems described herein are exemplary system configurations. Other configurations may exist. Those having skill in the art will appreciate that the invention described herein may work with various configurations. Accordingly, more or less of the aforementioned system components may be used and/or combined in various embodiments. It should also be understood that various software modules 115a-115n that are utilized to accomplish the functionalities described herein may be maintained on different components than controller 107, as desired or necessary. In other embodiments, as would be appreciated, the functionalities described herein may be implemented in various combinations of hardware and/or firmware, in addition to, or instead of, software. Furthermore, various operations of the methods described herein, while described in a particular order, may be performed in different orders as would be appreciated by those having skill in the art. In some embodiments, more of less of the described operations may be used.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A respiratory therapy device comprising:
a pressure generator that generates a flow of gas;
a delivery conduit connected to the pressure generator and having a patient interface, wherein the delivery conduit delivers the flow of gas to a patient connected to the patient interface;
a sensor integrated into the patient interface that measures blood oxygenation information for the patient; and
a controller that controls the pressure generator to manipulate one or more parameters of the flow of gas according to a positive pressure support therapy regime, receives the blood oxygenation information for the patient, and adjusts at least one of the one or more parameters based on the received blood oxygenation information.

2. The respiratory therapy device of claim 1, wherein the controller is configured such that adjusting at least one of the one or more parameters includes controlling the operation of the pressure generator to adjust a pressure of the flow of gas during inhalation and/or exhalation of the patient.

3. The respiratory therapy device of claim 1, wherein the controller is configured to advance a diagnosis of the patient based, at least in part, on the received blood oxygenation information.

4. The respiratory therapy device of claim 1, wherein the sensor is one of a reflectance-based sensor or a transmittance-based sensor.

5. The respiratory therapy device of claim 1, wherein the blood oxygenation information is the oxygen saturation of the patient's hemoglobin.

6. A method for providing respiratory therapy comprising:
generating a flow of gas with a pressure generator and providing the flow of gas to a patient using a patient interface of a respiratory therapy system;
measuring blood oxygenation information for the patient using a sensor integrated into the patient interface; and
manipulating one or more parameters of the flow of gas according to a positive pressure support therapy regime and adjusting at least one of the one or more parameters based on the received blood oxygenation information.

7. The method of claim 6, wherein adjusting at least one of the one or more parameters includes adjusting a pressure of the flow of gas during inhalation and/or exhalation of the patient.

8. The method of claim 6, further comprising advancing a diagnosis of the patient based, at least in part, on the received blood oxygenation information.

9. The method of claim 6, wherein measuring blood oxygenation information includes measuring the oxygenation information using one of a reflectance-based sensor or a transmittance-based sensor.

10. The method of claim 6, wherein the blood oxygenation information is the oxygen saturation of the patient's hemoglobin.

11. A respiratory therapy device comprising:
means for generating a flow of gas;
means for delivering the flow of gas to a patient via a patient interface;
means for measuring blood oxygenation information for the patient that is integrated into the patient interface; and
means for controlling the means for generating a flow of gas to manipulate one or more parameters of the flow of gas according to a positive pressure support therapy regime, receiving the blood oxygenation information for the patient, and adjusting at least one of the one or more parameters based on the received blood oxygenation information.

12. The respiratory therapy device of claim 11, wherein the means for controlling the means for generating a flow of gas is configured such that adjusting at least one of the one or more parameters includes controlling the operation of the means for generating a flow of gas to adjust a pressure of the flow of gas during inhalation and/or exhalation of the patient.

13. The respiratory therapy device of claim 11, wherein the means for controlling the means for generating a flow of gas is configured to advance a diagnosis of the patient based, at least in part, on the received blood oxygenation information.

14. The respiratory therapy device of claim 11, wherein the means for measuring blood oxygenation information is one of a reflectance-based sensor or a transmittance-based sensor.

15. The respiratory therapy device of claim 11, wherein the blood oxygenation information is the oxygen saturation of the patient's hemoglobin.
